(19) United States
(12) Patent Application Publication
(10) Pub. No.: US 2023/0253102 A1
(43) Pub. Date: Aug. 10, 2023

(54) SYSTEM AND METHOD FOR CARING FOR PATIENTS BY USING A SMART DIAPER

(71) Applicant: NC Co., LTD., Cheongdo-gun (KR)

(72) Inventors: Chun Gwang BYUN, Seoul (KR); Ji Hyun BYUN, Seoul (KR)

(21) Appl. No.: 17/863,476

(22) Filed: Jul. 13, 2022

(30) Foreign Application Priority Data
Feb. 6, 2022 (KR) 10-2022-0067690

(51) Int. Cl. G16H 40/63

(52) U.S. Cl. CPC G16H 40/63

(57) ABSTRACT

The present invention relates to a comprehensive patient care system and method using a smart diaper that provides a detection result in a smart diaper through a patient care dedicated app executed in a caregiver terminal. A comprehensive patient care system and method using a smart diaper includes: a smart diaper equipped with a composite sensor that detects the wearer's urination or defecation and components including stool and urine, including any one or more of the following: a biosensor, a temperature sensor, a humidity sensor, a capacitive sensor, or a gas sensor; a matching unit that receives the smart diaper identification information and the patient identification information through a patient-care app executed in the caregiver terminal, matches the smart diaper identification information and the patient identification information, and stores the matching information; a result-receiving unit for receiving a detection result from the complex sensor provided in the smart diaper; a database unit for classifying the detection result received by the result-receiving unit into patient identification information, organizing, and storing; a comprehensive patient care device, including an information-providing unit that provides the information arranged in the database in the database unit to a patient terminal or a caregiver terminal; a smart diaper hospital-integrated monitoring system that can efficiently manage the occurrence of stool and urine in places where a large number of patients exist, such as hospitals and nursing homes, through the comprehensive patient care system using smart diapers.

### CROSS-REFERENCE TO RELATED APPLICATION

This application claims priority to Korean Patent Application No. 10-2022-0067690 filed in the Korean Intellectual Property Office on Jun. 2, 2022, the disclosure of which is incorporated by reference herein in its entirety.

### TECHNICAL FIELD

The present invention relates to a system and method for comprehensive patient care using a smart diaper. In more detail, it relates to a comprehensive care system and method for a smart diaper product connected to a patient-care app on a caregiver terminal which provides patient health information.

### DISCUSSION OF RELATED ART

In general, diapers are used for the purpose of preventing urine leakage to the outside of the body by being mounted on wearers who cannot handle urinating on their own, such as infants, seriously ill patients, and dementia patients.

If the wearer's stool and urine are not treated within a certain period of time after the wearer has excreted bodily material into the diaper, the wearer may experience health and hygiene problems such as rash, yeast infections, urinary tract infections, or eczema due to the urine remaining on the body for long periods of time, so the diaper needs to be replaced promptly.

However, it is very cumbersome for guardians, caregivers, nurses, etc. to check in real time whether the diaper-wearer's stool and urine are excreted and to process it. In addition, the method of checking the diaper with the naked eye, smell, touch, and so on may be objectionable to both the wearer and the guardian. Furthermore, there is a problem that a small amount of urine is unsanitary because it is not easy to confirm.

In order to improve this problem, a functional diaper that is discolored has been developed with the purpose of informing caregivers of the presence or absence of urine during urination of the wearer.

In particular, smart diapers that are equipped with a detection sensor and the like to notify guardians of urination and urination as an alarm are also being continuously developed.

In a conventional smart diaper, a conductive detection line is embedded in the diaper at regular intervals, and an extension line of the detection line is connected to a detachable terminal made of magnetic material in a band.

In particular, urination is detected in such a way that the detector gets a change in the current of the detection line transmitted through the detachable terminal, confirms whether urination is present, and sends this to the speaker to generate an alarm melody.

However, since the conventional smart diaper is made of copper wire or a conductive paint is applied and used, it causes skin damage when it comes into contact with the wearer's body, has a poor fit, and is expensive to manufacture.

In addition, since most conventional smart diapers are focused on infants, research and development on a method of mounting a measuring device on an adult diaper have thus far been insufficient.

On the other hand, the timing, frequency, and cycle of urine experienced by severely ill patients and dementia patients can be important medical information, and it is almost impossible for guardians or nurses to check and record this information one by one at a hospital.

Furthermore, as the population is aging rapidly around the world and the prevalence of dementia of the elderly continues to increase, there is a constant need for a plan to control the location and health status of many patients in real time in hospitals, nursing hospitals, and nursing homes.

The above descriptions as the background technology are only for improving understanding of the background of the present invention and should not be accepted as acknowledging that they correspond to the conventional technology known to those skilled in the technology.

### SUMMARY

The present invention is derived from this technical background, and an object of the present invention is to provide a comprehensive patient care system and method using smart diapers, which may efficiently manage whether urine occurs in a plurality of patients such as a hospital and a nursing institution.

Another object of the present invention is to provide a comprehensive patient care system and method using a smart diaper capable of more detailed and accurate patient management by integrally transmitting monitored patient state information using a smart diaper to a medical institution server.

The present invention for achieving the above object includes the following configuration.

The comprehensive patient care system using a smart diaper, according to an embodiment of the present invention, includes at least one of the following sensors: a biosensor, a temperature sensor, a humidity sensor, a capacitive sensor, and a gas sensor.

It receives smart diaper identification and patient identification information through a smart diaper equipped with a complex sensor that detects the wearer's stool and urine and components, including urine and stool, and a patient care-only app running on a caregiver terminal.

Thereafter, it is divided into a matching unit that matches and stores smart diaper identification information and patient identification information, a result-receiving unit that receives a detection result from a complex sensor provided in the smart diaper, and patient identification information that receives the detection result received from the result-receiving unit. And it also covers a comprehensive patient care device, including a database unit for storing the data in a database and an information-providing unit for providing data-based information in the database unit to a patient terminal or a caregiver terminal.

The method performed in the comprehensive patient care system using smart diapers includes receiving smart diaper identification information and patient identification information through a patient-care app run by a caregiver terminal, matching and storing smart diaper identification information.

In addition, the result receiver is provided in the smart diaper, and the result is detected through a composite sensor that detects the wearer's stool and urine, including any one or more of a biosensor, a temperature sensor, a humidity sensor, a capacitive sensor, or a gas sensor.

Furthermore, this includes a data storage step of classifying and storing the received detection result into identification information for each patient and an information transmission step of providing the stored information to a patient terminal or a caregiver terminal.

The smart diaper presented by this invention provides a smart diaper integrated monitoring system that can efficiently manage the occurrence of stool and urine in hospitals and nursing homes where there are multiple patients. Furthermore, it provides a comprehensive patient care system and method using such a smart diaper.

In addition, the present invention has the effect of providing a system and method for comprehensive patient care using a smart diaper, which can expect more detailed and accurate patient management by transmitting the integrated information of the patient's status monitored using the smart diaper to the medical institution server.

### BRIEF DESCRIPTION OF THE DRAWINGS

FIG. 1 is an exemplary diagram explaining the comprehensive patient care system operation according to an embodiment of the present invention.

FIG. 2 is a block diagram illustrating the configuration of a comprehensive patient care apparatus according to an embodiment of the present invention.

FIG. 3 is a flowchart illustrating a method for comprehensive patient care according to an embodiment of the present invention.

### DETAILED DESCRIPTION

It should be noted that the technical terms used in the present invention are only used to describe specific embodiments and are not intended to limit the present invention. In addition, the technical terms used in the present invention should be interpreted as meanings generally understood by those of ordinary skill in the art to which the present invention pertains, unless otherwise specifically defined in the present invention. Accordingly, it should not be construed in an overly comprehensive sense or in an overly narrow sense. Hereinafter, preferred embodiments according to the present invention will be described in detail with reference to the accompanying drawings.

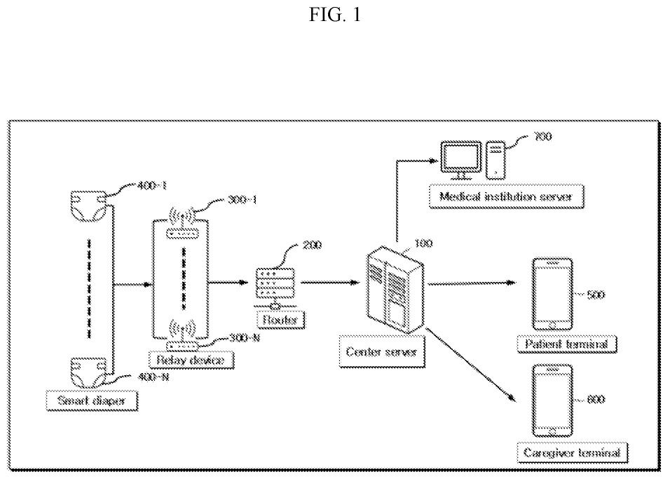

FIG. 1 is an exemplary diagram for explaining the operation of the comprehensive patient care system according to an embodiment of the present invention.

As shown in FIG. 1, the comprehensive patient care system according to an embodiment is mounted on one or more wearers, respectively, and receives data from multiple smart diapers (400-1, 400-2, . . . 400-N) that can detect the wearer's stool and urine, processes and displays data from multiple repeaters (300-1, 300-2, . . . 300-N) which receive data to aggregate the patient.

It is composed of a router (200) transmitted to the care device (100) and a comprehensive patient care system (100) that analyzes and stores data received from the router (200) and which can be accessed by a plurality of user terminals.

At this time, the relay devices (300-1, 300-2, . . . ,300-N) receive data of the smart diapers (400-1, 400-2, . . . , 400-N) in a certain area by grouping a plurality of smart diapers (400-1, 400-2, . . . , 400-N) according to a predetermined criterion, and the router (200) integrates data received from the plurality of relay devices (300-1, 300-2, . . . , 300-N) into a patient care device (100).

The smart diapers (400-1, 400-2, . . . 400-N) are, in one embodiment, disposed to surround the absorbent layer. However, each smart diaper includes a conductive member and further includes an absorption diffusion layer for diffusing the wearer's urination into the absorption layer.

The lining layer is arranged and positioned outside the absorption diffusion layer. The conductive sub-material included in the absorption diffusion layer is disposed to be in contact with the conductive sub-material included in the layer facing at least a portion of the absorbent layer, the lining layer, and the waterproof layer.

The conductive sub-material included in each lining layer, the absorption diffusion layer, the absorption layer, and the waterproof layer is, in a vertical direction, at least a portion of each of which is disposed at a position facing each other. Through this, it may be configured to be electrically connected to the complex sensor module disposed on the outer surface of the smart diaper.

And when the wearer's urination is absorbed into the inner surface of the smart diaper, the composite sensor module can sense the wearer's urination or defecation state by using a change in current through the conductive member.

In another embodiment, the smart diaper (400-1, 400-2, . . . 400-N) is worn on the patient so that the absorbent layer absorbs urine discharged from the patient and pulses on the opposite side of the urine-absorbing surface. It includes a conductor embodied as a conductive component.

It is also possible to apply a pulse to a smart diaper and a conductor in the smart diaper, sense a pulse induced from the conductor, and analyze the sensing result to determine whether it is urination or defecation in the diaper, as well as the amount of urination.

The complex sensor module includes an ultra-sensitive semiconductor sensor capable of detecting a disease-causing factor in excreted urine or stool.

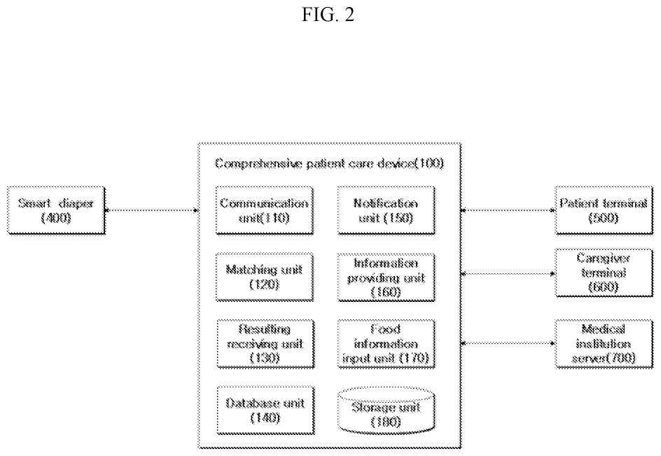

FIG. 2 is a block diagram illustrating the configuration of a comprehensive patient care system according to an embodiment of the present invention.

The comprehensive patient care system(100), according to an embodiment, may provide a comprehensive patient care technology capable of quickly and accurately checking and managing a patient's overall health condition or major diseases by introducing a smart diaper and using stool.

Furthermore, by utilizing the urine and urine detection data using smart diapers to control the patient's diet, it is possible to adjust the diet according to the patient's health condition, thereby enabling customized care for each patient.

In one embodiment, the comprehensive patient care system(100) embeds a small chip that can be linked with a smartphone app in the smart diaper (400) of the person to be treated, and manages temperature/humidity and other conditions from various biosensors mounted on the small chip. By collecting variable-related information, it is possible to inform the replacement cycle of the base station, and to manage a large number of persons to be treated based on the accumulated data.

Specifically, the smart diaper (400) is configured as follows by including any one or more of a temperature sensor, a humidity sensor, a capacitive sensor, and a gas sensor on a small chip.
i) A complex sensor that detects the wearer's stool and urine
ii) A motion sensor that detects the wearer's movement and generates angular level data
iii) A short-distance communication module that wirelessly transmits the data obtained from the complex sensor and the motion sensor to the repeaters (300-1, 300-2, . . . 300-N), and communicates with the satellite to measure the position coordinates of the current measuring device and a GPS module that performs calculations
iv) A control unit that analyzes the data obtained from the complex sensor and the motion sensor, and controls the activation of the short-distance communication module and the GPS module
v) A departure-prevention unit that notifies whether the wearer has deviated from a preset area according to the signal strength between the smart diaper (400) and the relay devices (300-1, 300-2, . . . 300-N)

The relay devices (300-1, 300-2, . . . 300-N) process the data received from the smart diaper (400) and process data information including the wearer's stool cycle, the wearer's sleep cycle, or the elapsed time after changing the diaper. It may include a data processing unit to operate with. However, the present invention is not limited thereto, and the comprehensive patient care system (100), according to an exemplary embodiment, may be configured by the smart diaper (400), the relay devices (300-1, 300-2, . . . 300-N), and the router (200). All signals can be received, analyzed, and processed.

As shown in FIG. 2, the comprehensive care device (100) for a person to be treated includes a communication unit (110), a matching unit (120), a result-receiving unit (130), a database unit (140), an information-providing unit (160), a notification unit (150), a food information input unit (170) and a storage unit (180).

The communication unit (110) communicates with any internal component or at least one external terminal through a wired/wireless communication network. The wireless Internet technology includes wireless LAN (WLAN), DLNA (Digital Living Network Alliance), WiBro (Wireless Broadband: Wibro), WiMAX (World Interoperability for Microwave Access: Wimax), HSDPA (High Speed Downlink Packet Access), High Speed Uplink Packet Access (HSUPA), IEEE 802.16, Long-Term Evolution (LTE), Long-Term Evolution-Advanced (LTE-A), Wireless Mobile Broadband Service (WMBS), and so on.

The communication unit (110) transmits and receives data according to at least one piece of wireless Internet technology within a range including Internet technologies not listed above.

In addition, short-distance communication technologies include Bluetooth, Radio Frequency Identification (RFID), Infrared Data Association (IrDA), Ultra Wideband (UWB), ZigBee, and Near Field Communication (NFC). These include Ultrasound Communication (USC), Visible Light Communication (VLC), Wi-Fi, Wi-Fi Direct, and the like. Furthermore, wired communication technologies include Power Line Communication (PLC), USB communication, Ethernet, serial communication, and optical/coaxial cables.

In an embodiment, the patient terminal (500) may be interpreted as a terminal possessed by a person to be treated, including a patient wearing the smart diaper (400). In this case, the persons to be cared for may include infants, children, elderly users with disorders such as dementia, and urinary incontinence patients.

The caregiver terminal (600) may be interpreted as a terminal possessed by a guardian or a caregiver who takes care of a patient who wears the smart diaper (400) or a nurse or doctor working in a hospital where the patient is being hospitalized. However, the present invention is not limited thereto. That is, anyone in charge of the care of the patient can become a caregiver. The matching unit (120) receives smart diaper identification information and patient identification information through an app exclusively for patient care executed in the caregiver terminal (600), matches the smart diaper identification information, and stores the same information in the storage unit (180).

In an aspect of the present invention, the matching unit (120) receives smart diaper identification information by recognizing the barcode or QR code printed on the smart diaper through an app dedicated to patient care executed in the caregiver terminal (600). Alternatively, the diaper identification information may be input by receiving the serial number printed on the smart diaper through the patient care dedicated app executed in the caregiver terminal (600).

A caregiver runs an app dedicated to patient care on the caregiver terminal (600) in their possession and recognizes by reading the QR code printed on the smart diaper that it needs to be replaced. Then, when patient information set in advance on the patient's app is selected and is input together, the matching unit (120) compares and stores the patient identification information and the smart diaper information worn by the patient.

Matching unit (120) may receive patient identification information by providing a user interface for selecting one of the patients being cared for by a caregiver or a caregiver through an app dedicated to patient care or by scanning a barcode or QR code including identification information of the caregiver printed on a bracelet.

According to an embodiment, the patient care-only app may provide a function of inputting or checking the overall care status for each of at least one or more patients to be treated by a caregiver or a guardian.

In addition, it provides a user interface for entering, registering, modifying, and deleting identification information of smart diapers or patient identification information using the dedicated patient-care app.

After displaying the nicknames or IDs for at least one patient for whom the caregiver or guardian serves, the patient care-only app can select one of them and present patient identification information.

The result receiving unit (130) is a device provided in the smart diaper (400), which includes any one or more of a biosensor, a temperature sensor, a humidity sensor, a capacitive sensor, and a gas sensor, receiving a detection result from a composite sensor that detects the wearer's urine and stool.

In this case, it is preferable that the “result-receiving unit” (130) receives the detection result together with the identification information of the corresponding smart diaper (400).

That is, the smart diaper (400) may apply a data processing technology for transmitting identification information of the corresponding smart diaper together with the detected result.

The database unit (140) divides the detection result received by the result-receiving unit (130) into the patient's identification information, forms a database, and stores it in the storage unit (180).

Specifically, the database unit (140) may identify the diaper identification information received together with the detection result in the result-receiving unit (130) and may identify the patient wearing the diaper based on the information stored in the matching unit (120).

That is, the database unit (140) stores the number of detections of urine for each patient and the patient's body temperature information into a database for each date or time zone. In an embodiment, the database unit (140) stores the number of stool and urine detections for each patient. In addition, the patient's body temperature information measured by the temperature sensor mounted on the smart diaper (400) may be stored in a database for each date or time zone. That is, it is possible to continuously monitor the number of stools and urine per day or changes in body temperature.

In addition, the database unit (140) may divide information for each patient and store information detected by various biosensors into a database. In this case, the database unit (140) may identify that the smart diaper (400) is equipped with different types of biosensors according to the identification information of the smart diaper (400). For example, it may be implemented to distinguish that different types of biosensors are mounted according to the identification information range of the smart diaper (400) displayed as letters or serial numbers displayed in specific digits of identification information.

The information-providing unit (160) provides the information databased in the database unit (140) to the patient terminal (500) or the caregiver terminal (600). In response to a request from the patient terminal (500) or the caregiver terminal (600), the information-providing unit (160) may provide information stored in the database unit (140) through a patient care dedicated app.

In an embodiment, the information-providing unit (160) may identify patient information matched to the smart diaper information based on information stored in the database unit (140).

In addition, the information-providing unit (160) may further determine the type of provided information set differently for each patient. For example, a certain patient may be set to be provided with information on the state of urine and stool, and other patients may be set to receive information about the state of body temperature. However, the present invention is not limited thereto and is construed to encompass various modifications.

The notification unit (150) transmits a notification message to the patient terminal or the caregiver terminal when the notification condition set differently for each patient is met based on the detection result received from the result-receiving unit (130) according to the patient identification information.

Accordingly, by introducing a smart diaper and using stool and urine, the patient's overall health status or major diseases can be checked and managed quickly and accurately.

At this time, the previously set notification condition is information input through the dedicated app for patient care executed in the caregiver terminal (600) and may be one of detection of urine and stool, whether the biosensor responds, and whether or not the setting area is deviated. However, the present invention is not limited thereto.

A notification message is transmitted according to a notification condition set differently for each patient through the dedicated app for patient care of the patient terminal (500) or the caregiver terminal (600).

For example, for patients who need to monitor stool and urine detection, the sending of notification messages according to urine detection is set up. In the case of patients who need to monitor whether or not to leave the setting area, a notification message on whether to leave the setting area can be sent.

That is, when it is necessary to manage a large number of patients, it is possible through the caregiver terminal (600) to individually set up the app to receive a notification message differently according to the respective behavioral state, thereby enabling customized care for the patient. In addition, it is implemented so that at least two or more caregiver terminals (600) can care for the same patient or person to be cared for. That is, a notification message may be provided to at least two or more caregiver terminals (600) according to the patient's individual behavioral state.

In one embodiment, whether the biosensor responds to one of such factors, including glucose, acidity (pH), protein, occult blood, urine specific gravity, bacteriuria, pyuria, and ketonuria, it may be implemented as a different voltage value output depending on whether the response is there or not.

In one aspect of the present invention, the result-receiving unit (130) is provided in the smart diaper (400) and receives the detection result from the ultra-sensitive semiconductor sensor that detects a factor that causes a previously set disease. At this time, the database unit (140) further stores the diagnosis information or nutritional status information based on the urine component into a database based on the detection result received by the result-receiving unit (130).

In other words, by introducing a smart diaper and using stool and urine, it is possible to provide comprehensive patient care that can quickly and accurately check and manage the patient's overall health condition or major diseases.

For example, the smart diaper (400) may be equipped with an ultra-sensitive semiconductor sensor capable of simultaneously measuring four prostate cancer factors (PSMA, ENG, ERG, ANXA3) contained in very trace amounts in urine. That is, the biosensor mounted on the smart diaper (400) can cover and apply various types of sensing technologies capable of detecting disease diagnosis or nutritional status based on urine components.

Accordingly, when it is necessary to manage a large number of persons to be treated, the caregiver can set a notification message for the response of different biosensors for each patient's condition, so that appropriate customized care is possible according to the health condition of the person to be treated.

In a further aspect of the present invention, the information-providing unit (160) transmits the detection result for each patient stored in the database unit (140) to a medical institution server (700) to provide more information.

In one embodiment, the information-providing unit (160) may provide the detected information of the biosensor for the urine component detected in the stool and urine of the person to be treated to the medical institution server (700) of the attending hospital of the person to be treated.

The information-providing unit (160) may periodically provide the accumulated health state detected through the smart diaper (400) for each caregiver to the medical institution server (700) or may extract and provide information stored in the storage unit (180) when a request for information on a specific caregiver is received from the medical institution.

That is, the accumulated data sensed using the smart diaper worn by the patient is shared with the hospital in charge of the patient or with the attending physician. Through this, the effect of enabling comprehensive and accurate management of patients through the data collected and analyzed from smart diapers in connection with the treatment system of the hospital is derived.

The food information input unit (170) receives dietary information and food intake information provided by the patient through the patient care dedicated app executed in the caregiver terminal (600). According to an embodiment, the patient care-only app provides a user interface through which the food intake status of the person to be treated can be easily input from a caregiver, that is, a caregiver or a guardian for each date and time unit. The database unit (140) further converts the food intake information input to the food information input unit (170) into a database and stores it. According to an embodiment, the database unit (140) divides each patient into a database and stores the food intake state of the person to be treated by date and time.

In addition, the information-providing unit (160) further provides the information stored in the database unit (140) to the medical institution server (700).

Also, the information-providing unit (160) may further provide the information on the patient's food intake status by date and time, which is stored in the database unit (140), to the patient terminal (500) or the caregiver terminal (600). Accordingly, for example, when a patient is hospitalized in a nursing facility, such as a nursing hospital, there is an effect that the guardian can accurately grasp the patient's food intake or stool status in realtime.

In other words, it is possible to monitor food intake and stool and urine status together and, based on the monitored data, take quick and appropriate actions when a patient-specific dietary adjustment is required, thereby effectively applying dietary adjustment to improve health. Additionally, the information-providing unit (160) may receive information about the type of medicine, injection, and dose prescribed by the patient through a dedicated patient-care app executed in the caregiver terminal (600).

That is, not only the state of stool and urine or food intake, but also the information about injections, types of drugs, and dosages prescribed by the patient in a nursing facility, such as a nursing hospital, is stored in a database.

Thereby, there is an effect of providing high-accuracy data that can help the patient's care when receiving treatment from a guardian of a patient who is hospitalized in a nursing facility or from another hospital or tertiary hospital.

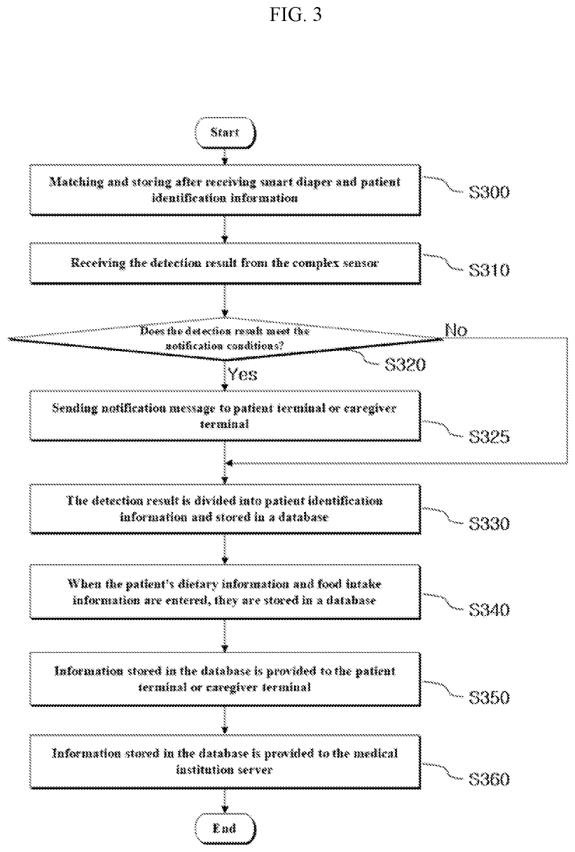

FIG. 3 is a flowchart illustrating a method of operating a comprehensive patient care system according to an embodiment of the present invention. In one embodiment, in the comprehensive patient care method performed with the comprehensive patient care device using the smart diaper, the matching unit receives smart diaper identification information and patient identification information through a dedicated patient care app executed on the caregiver terminal. This is stored by matching the smart diaper identification information with the patient identification information (S300).

In the characteristic aspect of the present invention, the matching and storing step recognizes the QR code printed on the smart diaper through a dedicated patient care app executed in the caregiver terminal and receives smart diaper identification information. Alternatively, the diaper identification information may be input by receiving the serial number printed on the smart diaper through the patient care-only app executed on the caregiver terminal.

A caregiver runs an app dedicated to patient care that recognizes the QR code printed on the smart diaper on the caregiver's terminal.

And when patient information set in advance on the patient's app is selected and input together, the matching unit matches and stores the patient identification information and the smart diaper information worn by the patient.

Furthermore, the result receiving unit obtains a detection result from a composite sensor that detects the wearer's stool and urine, including any one or more of the following: a biosensor, a temperature sensor, a humidity sensor, a capacitive sensor, or a gas sensor, which is provided in the smart diaper (S310). The result receiving unit preferably receives the detection result together with identification information of the corresponding smart diaper.

That is, a data processing technology that transmits the identification information of the smart diaper together with the detected result from the smart diaper.

In one aspect of the present invention, when the notification unit receives a measurement value that meets the notification condition, which is set differently for each patient according to the patient identification information based on the detection result received in the receiving step (S320), a notification message is sent to the patient terminal or the caregiver terminal (S325).

At this time, the set notification condition is information input through the patient care app running on the caregiver terminal, and may be one of stool and urine detection, biosensor response, and deviation from the setting area. However, the present invention is not limited thereto and should be construed to encompass various modifications.

The notification unit transmits a notification message according to a notification condition set differently for each patient through the patient care app on the patient terminal or the caregiver terminal.

For example, in the case of a patient who needs monitoring for detection of stool and urine, set the notification message according to detection of stool and urine. In the case of a patient who needs monitoring for deviation from the parameters set, it is possible to send a notification message about whether there has been deviation from those set parameters.

In other words, when management of multiple patients is required, it is possible to receive a different notification message according to each behavioral state through the caregiver terminal for each patient so that customized care for the patient is possible. Thereafter, the database unit divides the detection result received in the receiving step into patient identification information and stores it in a database (S330).

Specifically, the database unit may identify the received diaper identification information along with the detection result in the receiving step, and may determine the identification information from the patient wearing the corresponding diaper based on information stored in the matching unit.

In an embodiment, the database unit may record the number of stool and urine detections for each patient and the patient's body temperature information measured by the temperature sensor mounted on the smart diaper for each day or time period. In other words, it is possible to continuously monitor the amount of stool and urine per day or changes in body temperature.

The database unit may classify and store information detected by various biosensors into a database for each patient. In this case, the database unit may identify that the smart diaper is a smart diaper equipped with a different type of biosensor according to the identification information of the smart diaper.

For example, it may be implemented to distinguish that different types of biosensors are mounted according to the identification information range of the smart diaper displayed as alphabets or serial numbers displayed in specific digits of identification information. In another aspect of the present invention, the food information input unit receives dietary information and food intake information provided by the patient through the dedicated patient care app executed on the caregiver terminal (S340).

In database storage step, the food intake information received through the food information input unit is converted into a database record and stored.

And in the step of storing the information in a database by the information providing unit, the recorded information is provided to the patient terminal or the caregiver terminal (S350).

In addition, the information providing unit may further determine the type of information provided for each patient. For example, one patient can be set to receive information on stool and urine status, and another patient can be set to receive information about body temperature status.

In an embodiment, the app dedicated to patient care provides a user interface through which a patient's food intake status can be easily input by a caregiver or a guardian for each date and time unit.

Accordingly, for example, when a patient is hospitalized in a nursing facility, such as a nursing hospital, the guardian can accurately grasp the patient's food intake or stool and urine status in real-time.

The above-described method may be implemented as an application or implemented in the form of program instructions that may be executed through various computer components and recorded in a computer-readable recording medium. The computer-readable recording medium may include program instructions, data files, data structures, etc., alone or in combination.

The program instructions recorded on the computer-readable recording medium are specially designed and configured for the present invention and may be known and used by those skilled in the art of computer software.

Examples of computer-readable recording media include magnetic media such as hard disks, floppy disks, and magnetic tapes, optical recording media such as CD-ROM and DVD, magneto-optical media such as floptical disks, and hardware devices specifically configured to store and execute program instructions such as ROM, RAM, and flash memory.

Examples of program instructions include not only machine language codes such as those generated by a compiler, but also high-level language codes that can be executed by a computer using an interpreter or the like.

The hardware device may be configured to operate as one or more software modules for carrying out the processing according to the present invention and vice versa.

Although described above with reference to the embodiments, it will be understood by those skilled in the technologies that the present invention may be variously modified and changed within a range that does not deviate from the spirit and area of the present invention described in the following claims.

[Explanation of numbers]

| | |
|---|---|
| 100: Comprehensive patient care system | 110: Communication unit |
| 120: Matching unit | 130: Result receiving unit |
| 140: Database unit | 150: Notification unit |
| 160: Information providing unit | 170: Food information input unit |
| 180: Storage unit | 400: Smart diaper |
| 500: Patient's terminal | 600: Caregiver terminal |
| 700: Medical institution server | |

What is claimed is:

1. A smart diaper equipped with a composite sensor that detects the wearer's urination or defecation and components including stool and urine, including any one or more of: a biosensor, a temperature sensor, a humidity sensor, a capacitive sensor, or a gas sensor; a matching unit that receives the smart diaper identification information and the patient identification information through a patient care app executed on the caregiver terminal, matches the smart diaper identification information and the patient identification information, and stores the match information; a result receiving unit for receiving a detection result from the complex sensor provided in the smart diaper; a database unit for dividing the detection result received by the result receiving unit into patient identification information and storing it in a database; a comprehensive patient care device including an information providing unit that provides the databased information in the database unit to a patient terminal or a caretaker terminal; a comprehensive patient care system using a smart diaper including the above parts.

2. The smart diaper of claim 1, wherein the comprehensive patient care device is:
a comprehensive patient care system using a smart diaper including the following configuration: patient terminal or caregiver terminal. If different patient-specific notification conditions are met based on the patient identification information and based on the detection results received from the receiving unit, it includes notifications that send out notification messages; the notification conditions already set are information entered through an app dedicated to patient care running on the caregiver terminal; it includes stool and urine detection, response of the biosensor, and deviation from the settings.

3. The smart diaper of claim 1, wherein the matching unit is a comprehensive patient care system using smart diapers that recognizes QR codes printed on smart diapers through an app dedicated to patient care run by a caregiver terminal and inputs smart diaper identification information.

4. The smart diaper of claim 1, wherein the information providing unit corresponds to a comprehensive patient care system using a smart diaper that further provides patient-specific detection results organized in the database unit to a medical institution server selected by the patient on request.

5. The smart diaper of claim 4, wherein the comprehensive patient care device includes a food information input unit for receiving diet information and food intake information provided by a patient through an app exclusively for patient care executed on a caregiver terminal, and the database unit organizes and stores food intake information input by the food information input unit. In addition, the information providing unit includes a comprehensive patient care system using a smart diaper that further provides information stored in the database unit to a medical institution server.

6. The smart diaper of claim 1, wherein the result receiving unit is provided in the smart diaper and receives a detection result from an ultra-high-sensitivity semiconductor sensor that detects a factor causing a preset disease. In addition, the database unit includes a comprehensive patient care system using a smart diaper that organizes and stores diagnostic information or nutritional status information of a disease based on a urine component based on a detection result received by the result receiving unit.

7. The smart diaper of claim 1, wherein the database unit is a comprehensive patient care system using a smart diaper that organizes and stores the amount of stool and urine detected and body temperature information by date or time period.

8. A method of comprehensive patient care performed in a comprehensive patient care system using smart diapers includes the following items: the matching unit receives smart diaper identification and patient identification information through the patient care-only app run on the caregiver terminal, and matches and stores the smart diaper identification and patient identification information; the stage in which the result receiving unit receives the detection result from a combined sensor that detects the wearer's stool and urine, including at least one of the biosensors, temperature sensors, humidity sensors, capacitance sensors, or gas sensors in the smart diaper; a step in which the database unit divides the received detection results into patient identification information and stores them in a database; a step in which the information providing unit provides information on the organized information to the patient terminal or the caregiver terminal in the step in which the organized information is stored in the database; a comprehensive patient care method using smart diapers containing this content.

9. The method of claim 8, wherein the notification unit includes transmitting a notification message to the patient terminal or the caregiver terminal when the notification unit meets a notification condition, which is set differently for each patient according to the patient identification information based on the received detection result. The set notification condition is information input through an app dedicated to patient care executed on the caregiver terminal, including a comprehensive patient care method using a smart diaper, which is one of stool and urine detection, bio-sensor reaction, and deviation from set parameters.

10. The method of claim 8, wherein the matching and storing step includes a comprehensive patient care method using a smart diaper, which recognizes a QR code printed on a smart diaper through a patient care-only app executed on a caregiver terminal and receives smart diaper identification information.

11. The method of claim 8, wherein the providing of information includes a comprehensive patient care method using a smart diaper that further provides patient-specific detection results organized into databases at the time of storing the information, either at a request from a medical institution or periodically at a medical institution server selected by the patient.

12. The method of claim 11, wherein the food information input unit includes receiving diet information and food intake information provided by a patient through an app exclusively for patient care executed on a caregiver terminal, and the information organized and stored in the database is organized into a database. The step of providing the information includes a comprehensive patientcare method using a smart diaper, which further provides information organized in the step of storing it as the database to a medical institution server.
